(19) United States
(12) Patent Application Publication
(10) Pub. No.: US 2017/0027830 A1
(43) Pub. Date: Feb. 2, 2017

(54) HAIR DYE COMPOSITION

(71) Applicant: HOYU CO., LTD., Aichi (JP)

(72) Inventors: Jun UEHARA, Aichi (JP); Miyuki ENDO, Aichi (JP); Hiroyuki SUGAYA, Aichi (JP)

(21) Appl. No.: 15/224,334

(22) Filed: Jul. 29, 2016

(30) Foreign Application Priority Data
Jul. 31, 2015 (JP) 2015-151923

(51) Int. Cl. A61K 8/34; A61K 8/41

(52) U.S. Cl. CPC A61K 8/342; A61K 8/416; A61K 2800/4324; A61Q 5/10

(57) ABSTRACT

An object of the invention is to provide a hair dye composition containing an oxidation dye or a direct dye which has excellent emulsion stability. In order to achieve the object, a hair dye composition containing a surfactant having a sterol skeleton is provided. As a result, a hair dye composition containing an oxidation dye or a direct dye having excellent emulsion stability can be obtained.

### CROSS-REFERENCES TO RELATED APPLICATION

This application claims priority from Japanese Patent Application Serial No. 2015-151923 filed Jul. 31, 2015, the contents of which are incorporated herein by reference in its entirety.

### TECHNICAL FIELD

The present invention relates to a hair dye composition for dyeing hair or the like. More specifically, the invention relates to a hair dye composition which contains a dye and which has excellent emulsion stability.

### BACKGROUND ART

A hair dye composition for dyeing hair or the like contains an oily component such as a higher alcohol and silicone oil for example for the purposes of improving the applicability to the hair or the fastness to washing and improving the feeling of the hair such as smoothness. Such an oily component is added together with a surfactant and is contained in a hair dye composition as an emulsion. The oily component separates from other components of the emulsion with time, and thus it is required to improve the emulsion stability. In particular, it is known that the emulsion stability is apt to deteriorate when a dye is added.

For example, Japan Patent Application Publication No. 2014-47184 discloses that the emulsion stability of an aerosol foam-type oxidation hair dye composition containing an oxidation dye is improved by adding a non-ionic surfactant, a higher alcohol of C20 or more and a higher alcohol of C18 or less and adjusting the mass ratio of the non-ionic surfactant to the higher alcohols at 1.5 to 50.

Japan Patent Application Publication No. 2015-86175 discloses that, with respect to a cosmetic composition for hair, the emulsion stability of a first agent containing an oxidation dye as a salt is improved by adding polyethylene glycol having a number average molecular weight of 1290 or more and less than 7300 to the first agent.

Also, Japan Patent Application Publication No. 2002-226336, which is a patent document related to the invention, discloses a hair dye composition containing a direct dye and poly oxy ethylene(30) phytosterol.

### SUMMARY

An object of the invention is to provide a hair dye composition containing an oxidation dye or a direct dye which has excellent emulsion stability.

As a result of intensive investigation to achieve the object, the inventors have found that by adding a surfactant having a sterol skeleton to a hair dye composition containing an oxidation dye or a direct dye, a hair dye composition having excellent emulsion stability can be obtained. The inventors have thus completed the invention.

That is, the invention relates to a hair dye composition which is an emulsion composition containing an oily component, characterized by containing an oxidation dye (A-1) or a direct dye (A-2) and a surfactant having a sterol skeleton (B). According to the hair dye composition, a hair dye composition having excellent emulsion stability can be provided. Moreover, an improvement effect on the hair dyeing ability is also obtained because the surfactant having a sterol skeleton is added.

Furthermore, when the hair dye composition of the invention contains the oxidation dye (A-1), the hair dye composition is characterized by containing a lanolin derivative surfactant (C). Using the lanolin derivative surfactant (C) together with the surfactant having a sterol skeleton (B), particularly excellent emulsion stability can be obtained. Moreover, a particularly excellent effect on the hair dyeing ability is also obtained.

When the hair dye composition of the invention contains the direct dye (A-2), the hair dye composition is characterized by containing a cationic surfactant (D). By adding both of the surfactant having a sterol skeleton (B) and the cationic surfactant (D) to a hair dye composition containing the direct dye (A-2), excellent emulsion stability can be obtained. The hair dyeing ability is also improved.

The hair dye composition of the invention is also characterized in that the surfactant having a sterol skeleton (B) is polyoxyethylene phytosterol. Due to this characteristic, the effects of the invention can be exhibited better.

According to the invention, a hair dye composition which contains an oxidation dye or a direct dye and which has excellent emulsion stability can be provided.

Next, the invention including the best modes for carrying out the invention is explained. The hair dye composition of the invention is a hair dye containing an oxidation dye (A-1), a direct dye (A-2) or both and is applied to hair to deposit the oxidation dye or the direct dye on the hair or the inside thereof.

Hair dyes are classified into temporary hair dyes, semi-permanent hair dyes and permanent hair dyes depending on the durability of the effect of hair dyeing, namely the fastness. The hair dye composition of the invention may be used as any of these hair dyes. For example, the hair dye composition can be used for an oxidation hair dye containing an oxidation dye (a permanent hair dye), a color treatment composition containing a direct dye (a semi-permanent hair dye) and the like.

The hair dye composition of the invention is an emulsion composition containing an oily component and a surfactant. The type of the emulsion composition, for example an oil-in-water type, a water-in-oil type or the like, is not particularly limited, but an oil-in-water emulsion composition is preferable.

The hair dye composition of the invention may be in any form as long as it is an emulsion composition. Examples of the form include liquid, cream, gel and the like. In this regard, the hair dye composition should be an emulsion composition during the storage but may be converted into foam or mist at the time of the use. In the case of foam, an aerosol foamer container or a non-aerosol foamer container may be used.

Considering one of the effects of the invention, excellent emulsion stability namely, the hair dye composition is preferably in the form of emulsion liquid having a viscosity at 25° C. of 10,000 or less. With respect to the method for measuring the viscosity at 25° C., the viscosity is measured using a type B viscometer (manufactured by Toki Sangyo Co., Ltd) (rotor No. 3, 12 rpm, one minute).

The components contained in the hair dye composition of the invention are described in detail below. Unless otherwise specifically noted, the amounts of the components are the amounts in the hair dye composition during the storage. For example, in a two-part oxidation hair dye, the amounts are those in the first agent containing the oily component and the oxidation dye.

Oxidation Dye (A-1)

The oxidation dye of the invention is a dye which develops a color through oxidative polymerization caused by an oxidant. The types of oxidation dye include dye intermediates which develop a color by their own oxidation and couplers which produce various color tones depending on the combination with a dye intermediate.

Major dye intermediates are dye precursors which are o- or p-phenylenediamines or aminophenols and are generally colorless or slightly colored compounds. Specific examples thereof include p-phenylenediamine, toluene-2,5-diamine(p-toluylenediamine), N-phenyl-p-phenylenediamine, 4,4′-diaminodiphenylamine, p-aminophenol, o-aminophenol, p-methylaminophenol, N,N-bis(2-hydroxyethyl)-p-phenylenediamine, 2-hydroxyethyl-p-phenylenediamine, o-chloro-p-phenylenediamine, 4-amino-m-cresol, 2-amino-4-hydroxyethylaminoanisole, 2,4-diaminophenol, salts thereof and the like.

Major couplers are m-diamines, aminophenols or diphenols. Specific examples thereof include resorcin, catechol, pyrogallol, phloroglucin, gallic acid, hydroquinone, 5-amino-o-cresol, m-aminophenol, 5-(2-hydroxyethylamino)-2-methylphenol, m-phenylenediamine, 2,4-diaminophenoxyethanol, toluene-3,4-diamine, α-naphthol, 2,6-diaminopyridine, diphenylamine, 3,3′-iminodiphenyl, 1,5-dihydroxynaphthalene, tannic acid, salts thereof and the like.

The oxidation dye includes acid addition salts of the above compounds and the like. The acid addition salts are acid addition salts with inorganic acids such as hydrochlorides and sulfates and acid addition salts with organic acids such as acetates. It is preferable to blend an acid addition salt of an oxidation dye in view of the storage stability of the dye. Here, an acid addition salt of an oxidation dye may destabilize the emulsification. However, the hair dye composition of the invention exhibits high emulsion stability even when an acid addition salt of an oxidation dye is contained, and thus the storage stability of the dye and the emulsion stability of the hair dye composition can be both improved.

One, two or more of the oxidation dyes can be selected and used depending on the desired color tone. The amount of the oxidation dye added based on the hair dye composition is not particularly limited but is preferably 0.1 to 10 mass %, further preferably 1 to 5 mass %.

Direct Dye (A-2)

The direct dye of the invention is a compound having a color and is a dye which adheres to or penetrates through the hair to dye the hair. For example, the direct dye is an acid dye, a basic dye, a natural dye, a nitro dye, a HC dye, a disperse dye or the like. One of the direct dyes may be added alone, or a combination thereof may be added.

Examples of the acid dye include Acid Red 2, Acid Red 3, Acid Red 102, Acid Red 104 (1), Acid Red 105 (1), Acid Red 106, Acid Red 227, Acid Red 230 (1), Acid Yellow 4, Acid Yellow 5, Acid Yellow 202 (1), Acid Yellow 202 (2), Acid Yellow 203, Acid Orange 205, Acid Orange 207, Acid Orange 402, Acid Green 3, Acid Green 204, Acid Green 401, Acid Violet 401, Acid Blue 1, Acid Blue 2, Acid Blue 202, Acid Brown 201, Acid Black 401 and the like.

Examples of the basic dye include Basic Blue 3, Basic Blue 6, Basic Blue 7, Basic Blue 9, Basic Blue 26, Basic Blue 41, Basic Blue 47, Basic Blue 99, Basic Brown 4, Basic Brown 16, Basic Brown 17, Basic Green 1, Basic Green 4, Basic Orange 1, Basic Orange 2, Basic Orange 31, Basic Red 1, Basic Red 2, Basic Red 22, Basic Red 46, Basic Red 51, Basic Red 76, Basic Red 118, Basic Violet 1, Basic Violet 3, Basic Violet 4, Basic Violet 10, Basic Violet 11:1, Basic Violet 14, Basic Violet 16, Basic Yellow 11, Basic Yellow 28, Basic Yellow 57, Basic Yellow 87 and the like.

Examples of the natural dye include gardenia dye, turmeric dye, annatto dye, sodium copper chlorophyllin, paprika dye, lac dye, henna and the like.

Examples of the nitro dye include 4-nitro-o-phenylenediamine, 2-nitro-p-phenylenediamine, 2-amino-4-nitrophenol, 2-amino-5-nitrophenol, picramic acid, picric acid, salts thereof and the like.

Examples of the HC dye include HC Blue No. 2, HC Blue No. 5, HC Blue No. 6, HC Blue No. 9, HC Blue No. 10, HC Blue No. 11, HC Blue No. 12, HC Blue No. 13, HC Orange No. 1, HC Orange No. 2, HC Orange No. 3, HC Red No. 1, HC Red No. 3, HC Red No. 7, HC Red No. 10, HC Red No. 11, HC Red No. 13, HC Red No. 14, HC Violet No. 1, HC Violet No. 2, HC Yellow No. 2, HC Yellow No. 4, HC Yellow No. 5, HC Yellow No. 6, HC Yellow No. 9, HC Yellow No. 10, HC Yellow No. 11, HC Yellow No. 12, HC Yellow No. 13, HC Yellow No. 14, HC Yellow No. 15 and the like.

Examples of the disperse dye include Disperse Black 9, Disperse Blue 1, Disperse Blue 3, Disperse Blue 7, Disperse Brown 4, Disperse Orange 3, Disperse Red 11, Disperse Red 15, Disperse Red 17, Disperse Violet 1, Disperse Violet 4, Disperse Violet 15 and the like.

The amount of the direct dye added based on the hair dye composition is not particularly limited but is preferably 0.01 to 10 mass %, further preferably 0.1 to 3 mass %.

Oily Component

The oily component added to the hair dye composition of the invention is a component which is mixed with an aqueous component such as water or ethanol and a surfactant and which is emulsified. Examples thereof include a higher alcohol, fat or oil, wax, a hydrocarbon, a higher fatty acid, an ester, silicone oil, fluorine oil and the like. One, two or more of the oily components can be selected and used.

A higher alcohol is preferably an alcohol having eight carbon atoms or more. Examples thereof include cetyl alcohol (cetanol), stearyl alcohol, cetostearyl alcohol, oleyl alcohol, linoleyl alcohol, linolenyl alcohol, arachyl alcohol, behenyl alcohol, lauryl alcohol, myristyl alcohol, 2-hexyldecanol, isostearyl alcohol, 2-octyldodecanol, decyltetradecanol and the like.

Fat or oil is a triglyceride, namely a triester of a fatty acid and glycerin. Examples thereof include olive oil, rose hip oil, camellia oil, shea butter, macadamia nut oil, almond oil, tea seed oil, sasanqua oil, safflower oil, sunflower oil, soybean oil, cottonseed oil, sesame oil, beef tallow, cocoa butter, corn oil, peanut oil, rapeseed oil, rice bran oil, rice germ oil, wheat germ oil, adlay oil, grapeseed oil, avocado oil, carrot oil, castor oil, linseed oil, coconut oil, mink oil, egg yolk oil and the like.

Wax is an ester of a higher fatty acid and a higher alcohol. Examples thereof include beeswax, candelilla wax, carnauba wax, jojoba oil, lanolin, spermaceti wax, rice bran wax, sugarcane wax, palm wax, montan wax, cotton wax, bayberry wax, Chinese wax, kapok wax, shellac wax and the like.

A hydrocarbon is a compound consisting of carbon and hydrogen. Examples thereof include liquid paraffin, paraffin, microcrystalline wax, Vaseline, isoparaffin, ozocerite, ceresin, polyethylene, α-olefin oligomer, polybutene, synthetic squalane, squalene, hydrogenated squalane, limonene, turpentine oil and the like.

The higher fatty acid is preferably a fatty acid having eight carbon atoms or more. Examples thereof include lauric acid, myristic acid, palmitic acid, stearic acid, behenic acid, isostearic acid, hydroxystearic acid, 12-hydroxystearic acid, oleic acid, undecylenic acid, linoleic acid, ricinoleic acid, lanolin fatty acid and the like.

An ester is a compound obtained by dehydration reaction of a fatty acid and an alcohol. Examples thereof include diisopropyl adipate, 2-hexyldecyl adipate, isopropyl myristate, myristyl myristate, cetyl octanoate, cetyl isooctanoate, isononyl isononanoate, diisopropyl sebacate, isopropyl palmitate, 2-ethylhexyl palmitate, butyl stearate, isocetyl isostearate, hexyl laurate, decyl oleate, fatty acid (C10-30) (cholesterol/lanosterol esters), lauryl lactate, octyldodecyl lactate, acetylated lanolin, dipentaerythritol fatty acid esters, N-alkylglycol monoisostearate, lanolin derivative and the like.

Silicone oil is a synthetic polymer containing alternating silicon having an organic group and oxygen through chemical bonds. Examples thereof include dimethylpolysiloxane (INCI name: dimethicone), dimethylpolysiloxane having a hydroxy end group (INCI name: dimethiconol), methylphenylpolysiloxane, decamethylcyclopentasiloxane, dodecamethylcyclohexasiloxane, polyether-modified silicone, highly polymerized silicone having an average polymerization degree of 650 to 10000, amino-modified silicone, betaine-modified silicone, alkyl-modified silicone, alkoxy-modified silicone, mercapto-modified silicone, carboxy-modified silicone, fluorine-modified silicone and the like.

Of the above examples, examples of the amino-modified silicone include an aminopropylmethylsiloxane-dimethylsiloxane copolymer (INCI name: aminopropyl dimethicone), an aminoethylaminopropylsiloxane-dimethylsiloxane copolymer (INCI name: amodimethicone), an aminoethylaminopropylmethylsiloxane-dimethylsiloxane copolymer (INCI name: trimethylsilyl amodimethicone) and the like.

The amount of the oily component added is not especially limited. However, from the viewpoints of the improvement of the emulsion stability of the hair dye composition, the spreadability of the hair dye composition when applied to the hair and the like, the amount in a hair dye composition containing an oxidation dye is preferably in the range of 0.1 to 30 mass %, particularly preferably in the range of 1 to 20 mass %. The amount in a hair dye composition containing a direct dye is preferably in the range of 0.1 to 20 mass %, particularly preferably in the range of 1 to 15 mass %.

Surfactant

The hair dye composition of the invention is an emulsion composition obtained by emulsifying the oily component and an aqueous component using a surfactant, characterized by containing the surfactant having a sterol skeleton (B) as the surfactant. By adding the surfactant having a sterol skeleton (B), the emulsion stability which is deteriorated by the oxidation dye or the direct dye can be improved. Moreover, by adding the surfactant having a sterol skeleton (B), an improvement effect on the hair dyeing ability can also be given.

Surfactant Having a Sterol Skeleton (B)

The surfactant having a sterol skeleton is a surfactant having a sterol skeleton as a lipophilic group. The sterol skeleton is an alcohol having a cyclopenta hydrophenanthrene ring shown by the following formula 1 to which a hydroxy group is attached, and the surfactant having a sterol skeleton is a compound having the sterol skeleton to which a hydrophilic group is attached. The letters A to D in the following formula 1 indicate the rings constituting the cyclopenta hydrophenanthrene ring, and the numbers indicate the location numbers of the carbon atoms constituting the cyclopenta hydrophenanthrene ring.

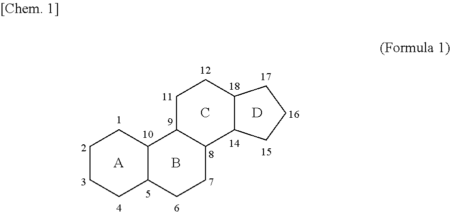

The sterol skeleton, which is a lipophilic group, may have a double bond in the cyclopenta hydrophenanthrene ring, and the position of the hydroxyl group is not especially limited. A sterol having a hydroxy group at the C-3 position and a double bond in the ring B or a stanol which has a hydroxy group at the C-3 position and which is constituted by saturated rings is preferable.

Examples of the sterol skeleton include phytosterols such as β-sitosterol, campesterol, stigmasterol and brassicasterol or stanols thereof, cholesterol, cholestanol, lanosterol, ergosterol and the like. Phytosterols and phytostanols are preferable.

The hydrophilic group may be of any structure. Examples thereof include non-ionic hydrophilic groups such as a polyoxyethylene chain, polyglycerin, polyglucoside, sorbitan and sucrose, anionic hydrophilic groups such as carboxylates, sulfonates, sulfates and phosphates and cationic hydrophilic groups such as aliphatic amine salts and quaternary ammonium salts. The hydrophilic group is preferably a non-ionic hydrophilic group, particularly preferably a polyoxyethylene chain. The addition number of moles of the polyoxyethylene chain is not particularly limited but is preferably 50 or less, more preferably 30 or less.

The HLB of the surfactant having a sterol skeleton is not particularly limited but is preferably 8 or more, further preferably 10 or more, particularly preferably 13 or more. In this regard, the HLB may be measured according to 20.3.1 Actual Measurement of HLB Value by Emulsification Method (pages 854 and 855) described in Handbook—Keshouhin/Seizai Genryou—Revised Edition (issued on Feb. 1, 1977, Nikko Chemicals Co., Ltd.). Hereinafter, in the present specification, the method for measuring HLB is according to this method.

Specific examples of the surfactant having a sterol skeleton include polyoxyethylene phytosterol, polyoxyethylene phytostanol, polyoxyethylene cholesterol, polyoxyethylene cholestanol and the like. Polyoxyethylene phytosterol and polyoxyethylene phytostanol are particularly preferable.

The amount of the surfactant having a sterol skeleton added is not particularly limited but is preferably 0.001 to 10 mass %, further preferably 0.01 to 5 mass %, particularly preferably 0.05 to 3 mass %.

Lanolin Derivative Surfactant (C)

When the hair dye composition of the invention contains the oxidation dye (A-1), the hair dye composition preferably further contains a lanolin derivative surfactant (C). When the lanolin derivative surfactant (C) is added together with the surfactant having a sterol skeleton (B) to a hair dye composition containing the oxidation dye (A-1), the emulsion stability and the hair dyeing ability of the hair dye composition containing the oxidation dye (A-1) can be improved synergistically.

Although the effect of the addition of the lanolin derivative surfactant (C) to a hair dye composition containing the oxidation dye (A-1) is described in the previous paragraph, this does not exclude the addition of the lanolin derivative surfactant (C) to a hair dye composition containing the direct dye (A-2).

Here, the lanolin derivative surfactant is a surfactant having a lanolin derivative as a lipophilic group. The lanolin derivative is a substance obtained by hydrolysis, hydrogenation, alkoxylation or the like from lanolin as a starting substance, and the lanolin derivative surfactant is a substance of the lanolin derivative to which a hydrophilic group is attached. Moreover, a substance in which the lanolin derivative itself has the surface-active property, such as potassium salts of lanolin fatty acids and sodium salts of lanolin fatty acids, may also be used.

The hydrophilic group attached to the lanolin derivative may be of any structure. Examples thereof include non-ionic hydrophilic groups such as a polyoxyethylene chain, polyglycerin, polyglucoside, sorbitan and sucrose, anionic hydrophilic groups such as carboxylates, sulfonates, sulfates and phosphates and cationic hydrophilic groups such as aliphatic amine salts and quaternary ammonium salts. The hydrophilic group is preferably a non-ionic hydrophilic group, particularly preferably a polyoxyethylene chain. The addition number of moles of the polyoxyethylene chain is not particularly limited but is preferably 50 or less, more preferably 30 or less.

The HLB of the lanolin derivative surfactant is not particularly limited but is preferably 8 or more, further preferably 10 or more, particularly preferably 13 or more.

Specific examples of the lanolin derivative surfactant include polyoxyethylene lanolin, polyoxyethylene lanolin alcohol, potassium salts of lanolin fatty acids, sodium salts of lanolin fatty acids and the like. Polyoxyethylene lanolin and polyoxyethylene lanolin alcohol are particularly preferable.

The amount of the lanolin derivative surfactant added is not particularly limited but is preferably 0.001 to 10 mass %, further preferably 0.01 to 5 mass %, particularly preferably 0.05 to 3 mass %.

Cationic Surfactant (D)

The hair dye composition of the invention preferably further contains a cationic surfactant (D) as a surfactant. When the cationic surfactant (D) is added together with the surfactant having a sterol skeleton (B) to a hair dye composition containing the direct dye (A-2), the emulsion stability and the hair dyeing ability of the hair dye composition containing the direct dye (A-2) can be improved synergistically. Also, when the cationic surfactant (D) is added together with the surfactant having a sterol skeleton (B) to a hair dye composition containing the oxidation dye (A-1), the hair dyeing ability of the hair dye composition containing the oxidation dye (A-1) can be further improved.

The cationic surfactant is a surfactant whose hydrophilic group becomes a cation when the surfactant dissolves in water and dissociates into ions. Examples thereof include alkyl quaternary ammonium salts such as monoalkyl quaternary ammonium salts, dialkyl quaternary ammonium salts, trialkyl quaternary ammonium salts, benzalkonium quaternary ammonium salts and monoalkyl ether quaternary ammonium salts, amine salts such as alkylamine salts, fatty acid amidoamine salts, ester-containing tertiary amine salts and Ahcovel tertiary amine salts, cyclic quaternary ammonium salts such as alkylpyridinium salts and alkyl isoquinolium salts, benzethonium chloride and the like.

The cationic surfactant is preferably an alkyl quaternary ammonium salt, further preferably a monoalkyl quaternary ammonium salt or a dialkyl quaternary ammonium salt, particularly preferably a monoalkyl quaternary ammonium salt.

Examples of the monoalkyl quaternary ammonium salt include lauryltrimethylammonium chloride, lauryltrimethylammonium bromide, alkyl(16,18)trimethylammonium chloride, cetyltrimethylammonium chloride, cetyltrimethylammonium bromide, cetyltrimethylammonium saccharin, stearyltrimethylammonium chloride, stearyltrimethylammonium bromide, stearyltrimethylammonium saccharin, alkyl(28)trimethylammonium chloride, diPOE(2)oleylmethyl ammonium chloride, diPOE stearylmethyl ammonium chloride, POE(1)POP(25)diethylmethyl ammonium chloride, POP methyldiethyl ammonium chloride and the like. Alkyl(16,18)trimethylammonium chloride and cetyltrimethylammonium chloride are particularly preferable. POE indicates a polyoxyethylene chain, and POP indicates a polyoxypropylene chain. A number in the brackets after POE or POP indicates the addition number of moles thereof. A number in the brackets after alkyl indicates the number of carbon atoms of the fatty acid chain. The same applies in the descriptions below.

Examples of the dialkyl quaternary ammonium salt include dialkyl(12-15)dimethylammonium chloride, dialkyl(12-18)dimethylammonium chloride, dialkyl(14-18)dimethylammonium chloride, dicocoyldimethylammonium chloride, dicetyldimethylammonium chloride, distearyldimethylammonium chloride, isostearyllauryldimethylammonium chloride and the like.

The amount of the cationic surfactant added is not particularly limited but is preferably 0.001 to 10 mass %, further preferably 0.01 to 5 mass %, particularly preferably 0.05 to 3 mass %.

Other Surfactant

A surfactant other than the surfactant having a sterol skeleton (B), the lanolin derivative surfactant (C) and the cationic surfactant (D) may be added to the hair dye composition of the invention. The other surfactant is a non-ionic surfactant, an anionic surfactant or an amphoteric surfactant.

Examples of the non-ionic surfactant include POE alkyl ethers, POE alkylphenyl ethers, POE•POP alkyl ethers, POE sorbitan fatty acid esters, POE monofatty acid esters, POE glycerin fatty acid esters, polyglycerin fatty acid esters, monoglycerin fatty acid esters, sorbitan fatty acid esters, sucrose fatty acid esters, alkyl polyglucosides and the like. Specific examples of the POE alkyl ethers include POE lauryl ether, POE cetyl ether, POE stearyl ether, POE behenyl ether and the like.

As the non-ionic surfactant, a non-ionic surfactant having HLB of 10 or less is preferably added. Specific examples of the non-ionic surfactant having HLB of 10 or less include, for example, POE(2)lauryl ether, POE(4)stearyl ether, POP(4)cetyl ether, POE(5)glyceryl monostearate and the like. A POE alkyl ether having HLB of 10 or less is particularly preferable.

The amount of the non-ionic surfactant added is not particularly limited but is preferably 0.001 to 40 mass %, further preferably 0.01 to 30 mass %, particularly preferably 0.05 to 20 mass %.

Specific examples of the anionic surfactant include alkyl sulfates such as sodium lauryl sulfate and triethanolamine lauryl sulfate, alkyl ether sulfates such as sodium POE lauryl ether sulfate, N-acyl methyl taurates such as sodium stearoyl methyl taurate, alkylbenzene sulfonates such as triethanolamine dodecylbenzene sulfonate, α-olefin sulfonates such as sodium tetradecene sulfonate and POE alkyl ether phosphates such as POE lauryl ether phosphate and salts thereof.

Specific examples of the amphoteric surfactant include alkyl diamino ethyl glycine hydrochlorides, sodium N-coconut oil fatty acid acyl-N′-carboxyethyl-N′-hydroxyethylethylenediamine, disodium N-coconut oil fatty acid acyl-N′-carboxyethoxyethyl-N′-carboxyethylethylenediamine, sodium palm oil fatty acid acyl-N-carboxyethyl-N-hydroxyethylethylenediamine, sodium lauryl amino propionate, sodium lauryl amino dipropionate, triethanolamine lauryl amino propionate, coconut oil alkyl betaine, lauryldimethylaminoacetic acid betaine, myristyldimethylaminoacetic acid betaine, stearyldimethylaminoacetic acid betaine, sodium stearyldimethylbetaine, coconut oil fatty acid amidopropyl betaine, lauric acid amide propyl betaine, lauryl hydroxysulfobetaine and the like.

The total amount of all the surfactants added is not particularly limited but is preferably 0.01 to 40 mass %, further preferably 0.05 to 35 mass %, particularly preferably 0.1 to 30 mass %.

Other Components

Other optional components can be added to the hair dye composition of the invention. Examples thereof include alkali agents such as ammonia and monoethanolamine, antioxidants such as ascorbic acid and anhydrous sodium sulfite, antiseptics such as phenoxyethanol and sodium benzoate, organic solvents such as ethanol, sugar such as sorbitol and maltose, water-soluble polymers such as hydroxyethyl cellulose and carboxyvinyl polymer, cationic water-soluble polymers such as poly-dimethylmethylene piperidinium chloride solution and diallyldimethylammonium chloridethydroxyethyl cellulose, polyhydric alcohols such as polyethylene glycol and dipropylene glycol, chelating agents such as trisodium ethylenediamine hydroxyethyl triacetate dihydrate and tetrasodium hydroxyethane diphosphonate solution, inorganic salts such as sodium chloride and sodium carbonate, pH adjusting agents such as lactic acid and ammonium hydrogen carbonate, hair restorer components, plant extracts, crude drug extracts, amino acids/peptides, urea, vitamins, fragrances and ultraviolet absorbers.

### EXAMPLES

The invention is explained specifically below using Examples, but the technical scope of the invention is not limited by the Examples.

Preparation of Hair Dye Compositions

(Hair Dye Compositions Containing Oxidation Dyes)

As hair dye compositions containing oxidation dyes, first agents of two-part oxidation hair dyes were prepared. The compositions of the first agents are shown in Tables 1 to 4 below. Each first agent was prepared as follows. Each components except for a dye solution obtained by dissolving the components A-1 in a part of purified water and ammonia were mixed under a condition at 80° C., thereby obtaining an emulsion. Next, the emulsion was cooled to normal temperature while stirring the emulsion with a propeller agitator, and the dye solution in which the components A-1 were dissolved and ammonia were added.

(Hair Dye Compositions Containing Direct Dye)

As hair dye compositions containing a direct dye, color treatment compositions were prepared. The compositions of the color treatment compositions are shown in Table 5 below. Each color treatment composition was prepared as follows. The components were mixed under a condition at 80° C., and an emulsion was thus obtained. Then, the emulsion was cooled to room temperature while stirring the emulsion with a propeller agitator.

Evaluation Methods

<Emulsion Stability>

A transparent standard bottle No. 4 (volume of about 37 mL) was filled about eight-tenths full with a hair dye composition and sealed hermetically. This was left still in an incubator at 50° C., and the state of the emulsion was evaluated visually after 24 hours. The criteria of the evaluation were as follows: a score of 5 was given when the emulsion scarcely separated; a score of 4 was given when the emulsion did not separate very much; a score of 3 was given when the emulsion separated slightly; a score of 2 was given when the emulsion separated to some degree; and a score of 1 was given when the emulsion separated considerably. The results of the evaluation are shown in Tables 1 to 3 and 5.

<Hair Dyeing Ability>

(Hair Dye Treatment Method Using Hair Dye Compositions Containing Oxidation Dye)

An oxidation hair dye was prepared by mixing the first agent and the second agent described below at 1:1. One gram of the oxidation hair dye was applied to 1 g of a hair-bundle and left at 30° C. for 30 minutes. Then, the hair-bundle was washed with shampoo to wash off the oxidation hair dye. Next, a conditioner was applied to the hair-bundle. The hair-bundle was rinsed to wash off the conditioner, and then the water was wiped off the hair-bundle with a towel. The treatment was finished by drying the hair-bundle with a dryer. The formulation of the second agent is as follows.

(Second Agent)

| | |
|---|---|
| cetanol | 1.2 mass % |
| POE(30)cetyl ether | 0.3 mass % |
| POE(10)cetyl ether | 0.3 mass % |
| stearyltrimethyl ammonium chloride | 1.0 mass % |
| hydrogen peroxide | 16.6 mass % |
| hydroxyethane diphosphonic acid | 0.1 mass % |
| 4Na hydroxyethane diphosphonate | 0.2 mass % |
| phenoxyethanol | 0.1 mass % |
| purified water | balance |

(Hair Dye Treatment Method Using Hair Dye Compositions Containing Direct Dye)

One gram of a hair-bundle was wet with water, and the water was wiped off with a towel. Then, 1 g of the color treatment composition was applied to the hair-bundle and left at 30° C. for 10 minutes. Then, the hair-bundle was rinsed to wash off the color treatment composition. Next, the water was wiped off the hair-bundle with a towel, and the treatment was finished by drying the hair-bundle with a dryer.

(Evaluation Method of Hair Dyeing Ability)

The states of dyeing of the hair-bundles dyed as described above were evaluated visually. The criteria of the evaluation were: a score of 5 for “particularly excellent”; a score of 4 for “excellent”; a score of 3 for “good”; a score of 2 for “somewhat bad”; and a score of 1 for “bad”. The results of the evaluation are shown in Table 4 (hair dye compositions containing oxidation dyes) and in Table 5 (hair dye compositions containing a direct dye).

TABLE 1

| | | | Comparative |
|---|---|---|---|
| Name of Component | | Example 1-1 | Example 1-1 |
| oily | Cetanol | 1 | 1 |
| component | behenyl alcohol | 0.5 | 0.5 |
| A-1 | p-phenylenediamine | 2 | 2 |
| | p-aminophenol | 1 | 1 |
| | 5-amino-o-cresol | 0.4 | 0.4 |
| | 2,4-diaminophenoxyethanol | 0.4 | 0.4 |
| | hydrochloride | | |
| B | polyoxyethylene phytosterol | 0.1 | — |
| | (20E.O.) | | |
| C | polyoxyethylene lanolin | 0.5 | 0.5 |
| | (30E.O.) | | |
| D | cetyltrimethylammonium | 1 | 1 |
| | chloride solution | | |
| | polyoxyethylene lauryl ether | 1 | 1 |
| | (2E.O.) | | |
| | polyoxyethylene cetyl ether | 1 | 1 |
| | (30E.O.) | | |

| | | |
|---|---|---|
| ascorbic acid | 0.3 | 0.3 |
| polyethylene glycol 400 | 2 | 2 |
| dipropylene glycol | 2 | 2 |
| trisodium ethylenediamine hydroxyethyl | 0.2 | 0.2 |
| triacetate dihydrate | | |
| anhydrous sodium sulfite | 0.2 | 0.2 |
| poly-dimethylmethylene piperidinium | 0.2 | 0.2 |
| chloride solution | | |
| strong ammonia water | 1.5 | 1.5 |
| monoethanolamine solution (70%) | 8.5 | 8.5 |
| ammonium hydrogen carbonate | 0.5 | 0.5 |
| Urea | 1 | 1 |
| fragrance | 0.2 | 0.2 |
| purified water | balance | balance |
| total | 100 | 100 |
| emulsion stability | 5 | 1 |

It is seen from Table 1 that extremely high emulsion stability was observed in Example 1-1 which contained polyoxyethylene phytosterol (B) as compared to Comparative Example 1-1 which did not contain the component (B). Accordingly, it can be considered that the addition of polyoxyethylene phytosterol (B) to a hair dye composition containing an oxidation dye has an improvement effect on the emulsion stability.

Although no Example is shown here, the emulsion stability due to the component (B) was observed also when toluene-2,5-diamine, resorcinol, m-aminophenol, α-naphthol and 1,5-dihydroxynaphthalene were used as the component (A-1).

TABLE 2

| | | Example | Example | Example | Example | Example | Example | Example |
|---|---|---|---|---|---|---|---|---|
| Name of Component | | 2-1 | 2-2 | 2-3 | 2-4 | 2-5 | 2-6 | 2-7 |
| oily | Cetanol | 1 | 1 | 1 | 1 | 1 | 1 | 1 |
| component | behenyl alcohol | 0.5 | 0.5 | 0.5 | 0.5 | 0.5 | 0.5 | 0.5 |
| A-1 | p-phenylenediamine | 0.8 | 0.8 | 0.8 | 0.8 | 0.8 | 0.8 | 0.8 |
| | p-aminophenol | 0.5 | 0.5 | 0.5 | 0.5 | 0.5 | 0.5 | 0.5 |
| | 5-amino-o-cresol | 0.2 | 0.2 | 0.2 | 0.2 | 0.2 | 0.2 | 0.2 |
| | 2,4-diaminophenoxyethanol hydrochloride | 0.2 | 0.2 | 0.2 | 0.2 | 0.2 | 0.2 | 0.2 |
| B | polyoxyethylene phytosterol (20E.O.) | 0.1 | — | 0.1 | 0.1 | 0.1 | 0.1 | 0.1 |
| | polyoxyethylene phytostanol (25E.O.) | — | 0.1 | — | — | — | — | — |
| C | polyoxyethylene lanolin (30E.O.) | 0.5 | 0.5 | — | 0.5 | — | 0.5 | 0.5 |
| | polyoxyethylene lanolin alcohol (40E.O.) | — | — | 0.1 | — | — | — | — |
| D | alkyltrimethylammonium chloride solution | 1 | 1 | 1 | — | — | — | 1 |
| | cetyltrimethylammonium chloride solution | — | — | — | 1 | 0.5 | — | — |
| | polyoxyethylene lauryl ether (2E.O.) | 1 | 1 | 1 | 1 | 1.5 | 2 | 0.5 |
| | polyoxyethylene cetyl ether (30E.O.) | 1 | 1 | 1 | 1 | 1 | 1 | 1 |

| | | | | | | | |
|---|---|---|---|---|---|---|---|
| ascorbic acid | 0.3 | 0.3 | 0.3 | 0.3 | 0.3 | 0.3 | 0.3 |
| polyethylene glycol 400 | 2 | 2 | 2 | 2 | 2 | 2 | 2 |
| dipropylene glycol | 2 | 2 | 2 | 2 | 2 | 2 | 2 |
| trisodium ethylenediamine hydroxyethyl triacetate dihydrate | 0.2 | 0.2 | 0.2 | 0.2 | 0.2 | 0.2 | 0.2 |
| anhydrous sodium sulfite | 0.2 | 0.2 | 0.2 | 0.2 | 0.2 | 0.2 | 0.2 |
| poly-dimethylmethylene piperidinium chloride solution | 0.2 | 0.2 | 0.2 | 0.2 | 0.2 | 0.2 | 0.2 |
| strong ammonia water | 1.5 | 1.5 | 1.5 | 1.5 | 1.5 | 1.5 | 1.5 |
| monoethanolamine solution (70%) | 8.5 | 8.5 | 8.5 | 8.5 | 8.5 | 8.5 | 8.5 |
| ammonium hydrogen carbonate | 0.5 | 0.5 | 0.5 | 0.5 | 0.5 | 0.5 | 0.5 |
| Urea | 1 | 1 | 1 | 1 | 1 | 1 | 1 |
| Fragrance | 0.2 | 0.2 | 0.2 | 0.2 | 0.2 | 0.2 | 0.2 |
| purified water | balance | balance | balance | balance | balance | balance | balance |
| Total | 100 | 100 | 100 | 100 | 100 | 100 | 100 |
| emulsion stability | 5 | 5 | 5 | 5 | 3 | 5 | 5 |

In Table 2, the kinds of the surfactant having a sterol skeleton (B), the lanolin derivative surfactant (C) and the cationic surfactant (D) were investigated. It is seen from Example 2-1 and Example 2-2 that also when polyoxyethylene phytostanol was used, emulsion stability equivalent to that of polyoxyethylene phytosterol was observed. Moreover, it is understood from Example 2-1, Example 2-3 and Example 2-4 that the emulsion stability did not change even when the kind of the lanolin derivative surfactant (C) and the kind of the cationic surfactant (D) were changed.

Furthermore, the effect of the combination of the component (B), the component (C) and the component (D) was investigated in Table 2. It is seen from Example 2-5 to Example 2-7 that the score of the emulsion stability of Example 2-5 which did not contain the component (C) was 3 and was lower than that of Example 2-1 to some degree. On the other hand, the scores of the emulsion stability were maintained high in Example 2-6 which did not contain the component (D) and in Example 2-7 in which the amount of the other non-ionic surfactant was reduced. The results suggest that the emulsion stability is improved specifically when the component (B) and the component (C) are combined.

TABLE 3

| | | Example | Example | Example | Example | Example |
|---|---|---|---|---|---|---|
| Name of Component | | 2-1 | 3-1 | 3-2 | 4-1 | 4-2 |
| oily | Cetanol | 1 | 1 | 1 | 1 | 1 |
| component | behenyl alcohol | 0.5 | 0.5 | 0.5 | 0.5 | 0.5 |
| A-1 | p-phenylenediamine | 0.8 | 0.8 | 0.8 | 0.8 | 0.8 |
| | p-aminophenol | 0.5 | 0.5 | 0.5 | 0.5 | 0.5 |
| | 5-amino-o-cresol | 0.2 | 0.2 | 0.2 | 0.2 | 0.2 |
| | 2,4-diaminophenoxyethanol | 0.2 | 0.2 | 0.2 | 0.2 | 0.2 |
| | hydrochloride | | | | | |
| B | polyoxyethylene phytosterol | 0.1 | 0.01 | 5 | 0.1 | 0.1 |
| | (20E.O.) | | | | | |
| C | polyoxyethylene lanolin | 0.5 | 0.5 | 0.5 | 0.1 | 5 |
| | (30E.O.) | | | | | |
| D | alkyltrimethylammonium | 1 | 1 | 1 | 1 | 1 |
| | chloride solution | | | | | |
| | polyoxyethylene lauryl ether | 1 | 1 | 1 | 1 | 1 |
| | (2E.O.) | | | | | |
| | polyoxyethylene cetyl ether | 1 | 1 | 1 | 1 | 1 |
| | (30E.O.) | | | | | |

| | | | | | |
|---|---|---|---|---|---|
| ascorbic acid | 0.3 | 0.3 | 0.3 | 0.3 | 0.3 |
| polyethylene glycol 400 | 2 | 2 | 2 | 2 | 2 |
| dipropylene glycol | 2 | 2 | 2 | 2 | 2 |
| trisodium ethylenediamine hydroxyethyl | 0.2 | 0.2 | 0.2 | 0.2 | 0.2 |
| triacetate dehydrate | | | | | |
| anhydrous sodium sulfite | 0.2 | 0.2 | 0.2 | 0.2 | 0.2 |
| poly-dimethylmethylene piperidinium | 0.2 | 0.2 | 0.2 | 0.2 | 0.2 |
| chloride solution | | | | | |
| strong ammonia water | 1.5 | 1.5 | 1.5 | 1.5 | 1.5 |
| monoethanolamine solution (70%) | 8.5 | 8.5 | 8.5 | 8.5 | 8.5 |
| ammonium hydrogen carbonate | 0.5 | 0.5 | 0.5 | 0.5 | 0.5 |
| Urea | 1 | 1 | 1 | 1 | 1 |
| Fragrance | 0.2 | 0.2 | 0.2 | 0.2 | 0.2 |
| purified water | balance | balance | balance | balance | balance |
| Total | 100 | 100 | 100 | 100 | 100 |
| emulsion stability | 5 | 5 | 5 | 5 | 4 |

In Table 3, the amounts of the component (B) and the component (C) were investigated. It is seen from Example 3-1 and Example 3-2 that the emulsion stability was at the equivalent level when the amounts of the component (B) were 0.01 to 5 mass %. From Example 4-1 and Example 4-2, it is seen that high emulsion stability was observed when the amount of the component (C) was 0.1 mass % while the emulsion stability deteriorated slightly when the amount was 5 mass % as compared to Example 2-1 in which 0.5 mass % of the component (C) was added.

TABLE 4

| | | | | | | | Comparative |
|---|---|---|---|---|---|---|---|
| Name of Component | | Example 5-1 | Example 5-2 | Example 5-3 | Example 5-4 | Example 5-5 | Example 2-1 |
| oily | Cetanol | 1 | 1 | 1 | 1 | 1 | 1 |
| component | behenyl alcohol | 0.5 | 0.5 | 0.5 | 0.5 | 0.5 | 0.5 |
| A-1 | p-phenylenediamine | 0.8 | 0.8 | 0.8 | 0.8 | 0.8 | 0.8 |
| | p-aminophenol | 0.5 | 0.5 | 0.5 | 0.5 | 0.5 | 0.5 |
| | 5-amino-o-cresol | 0.2 | 0.2 | 0.2 | 0.2 | 0.2 | 0.2 |
| | 2,4-diaminophenoxyethanol hydrochloride | 0.2 | 0.2 | 0.2 | 0.2 | 0.2 | 0.2 |
| B | polyoxyethylene phytosterol (20E.O.) | 0.1 | — | 0.01 | 5 | 0.1 | — |
| | polyoxyethylene phytostanol (25E.O.) | — | 0.1 | — | — | — | — |
| C | polyoxyethylene lanolin (30E.O.) | 0.5 | 0.5 | 0.5 | 0.5 | 0.5 | 0.5 |
| D | alkyltrimethylammonium chloride solution | 1 | 1 | 1 | 1 | — | 1 |
| | polyoxyethylene lauryl ether (2E.O.) | 1 | 1 | 1 | 1 | 1 | 1 |
| | polyoxyethylene cetyl ether (30E.O.) | 1 | 1 | 1 | 1 | 2 | 1 |

| | | | | | | |
|---|---|---|---|---|---|---|
| ascorbic acid | 0.3 | 0.3 | 0.3 | 0.3 | 0.3 | 0.3 |
| polyethylene glycol 400 | 2 | 2 | 2 | 2 | 2 | 2 |
| dipropylene glycol | 2 | 2 | 2 | 2 | 2 | 2 |
| trisodium ethylenediamine hydroxyethyl triacetate | 0.2 | 0.2 | 0.2 | 0.2 | 0.2 | 0.2 |
| dehydrate | | | | | | |
| anhydrous sodium sulfite | 0.2 | 0.2 | 0.2 | 0.2 | 0.2 | 0.2 |
| poly-dimethylmethylene piperidinium chloride solution | 0.2 | 0.2 | 0.2 | 0.2 | 0.2 | 0.2 |
| strong ammonia water | 1.5 | 1.5 | 1.5 | 1.5 | 1.5 | 1.5 |
| monoethanolamine solution (70%) | 8.5 | 8.5 | 8.5 | 8.5 | 8.5 | 8.5 |
| ammonium hydrogen carbonate | 0.5 | 0.5 | 0.5 | 0.5 | 0.5 | 0.5 |
| Urea | 1 | 1 | 1 | 1 | 1 | 1 |
| Fragrance | 0.2 | 0.2 | 0.2 | 0.2 | 0.2 | 0.2 |
| purified water | balance | balance | balance | balance | balance | Balance |
| Total | 100 | 100 | 100 | 100 | 100 | 100 |
| hair dyeing ability | 5 | 5 | 5 | 4 | 4 | 1 |

In Table 4, the results of the evaluation of the hair dyeing abilities of the hair dye compositions containing oxidation dyes are summarized. It is seen from Example 5-1 and Comparative Example 2-1 that the addition of the component (B) has an improvement effect on the hair dyeing ability, as in the evaluation of the emulsion stability. An equivalent hair dyeing ability was observed also in Example 5-2 in which a different component (B) was used.

In Example 5-3 and Example 5-4, the amount of the component (B) was investigated. A high hair dyeing ability was observed when the amount of the component (B) was 0.01 mass % while the hair dyeing ability deteriorated slightly when the amount was 5 mass % as compared to that of Example 5-1 in which 0.1 mass % of the component (B) was added.

Moreover, the hair dyeing ability of a hair dye composition which did not contain the component (D) was evaluated in Example 5-5. As a result, the hair dyeing ability deteriorated when the component (D) was not added. This suggests that the component (D) improves the hair dyeing ability when added together with the component (B).

TABLE 5

| | | | | Comparative | Comparative | Comparative | Comparative |
|---|---|---|---|---|---|---|---|
| Name of Component | | Example 6-1 | Example 6-2 | Example 3-1 | Example 3-2 | Example 3-3 | Example 3-4 |
| oily component | Silicone | 0.5 | 0.5 | 0.5 | 0.5 | 0.5 | 0.5 |
| | mineral oil | 4 | 4 | 4 | 4 | 4 | 4 |
| | Beeswax | 0.3 | 0.3 | 0.3 | 0.3 | 0.3 | 0.3 |
| | Vaseline | 0.5 | 0.5 | 0.5 | 0.5 | 0.5 | 0.5 |
| | Cetanol | 2.7 | 2.7 | 2.7 | 2.7 | 2.7 | 2.7 |
| | stearyl alcohol | 4 | 4 | 4 | 4 | 4 | 4 |
| A-2 | Basic Brown 16 | 0.5 | — | 0.5 | 0.5 | 0.5 | 0.5 |
| | HC Blue 2 | — | 0.5 | — | — | — | — |
| B | polyoxyethylene phytosterol | 0.1 | 0.1 | — | 0.1 | 0.1 | 0.1 |
| | (20E.O.) | | | | | | |
| D | alkyltrimethylammonium | 0.2 | 0.2 | 0.2 | — | — | — |
| | chloride solution | | | | | | |
| | POE lauryl ether (2E.O.) | 1 | 1 | 1.1 | 1.2 | 1 | 1.2 |

| | | | | | | |
|---|---|---|---|---|---|---|
| poly-dimethylmethylene piperidinium chloride | — | — | — | — | 0.2 | 0.2 |
| solution | | | | | | |
| hydroxyethyl cellulose | 0.5 | 0.5 | 0.5 | 0.5 | 0.5 | 0.5 |
| Phenoxyethanol | 0.5 | 0.5 | 0.5 | 0.5 | 0.5 | 0.5 |
| Fragrance | 0.3 | 0.3 | 0.3 | 0.3 | 0.3 | 0.3 |
| lactic acid (50%) | 0.01 | 0.01 | 0.01 | 0.01 | 0.01 | 0.01 |
| tetrasodium hydroxyethane diphosphonate | 0.1 | 0.1 | 0.1 | 0.1 | 0.1 | 0.1 |
| solution | | | | | | |
| purified water | balance | balance | balance | balance | balance | balance |
| Total | 100 | 100 | 100 | 100 | 100 | 100 |
| emulsion stability | 5 | 5 | 1 | 1 | 1 | 1 |
| hair dyeing ability | 5 | 5 | 1 | 1 | 1 | 1 |

In Table 5, the evaluation of the emulsion stabilities and the hair dyeing abilities of the hair dye compositions containing a direct dye are summarized. It is seen from Example 6-1, Comparative Example 3-1 and Comparative Example 3-2 that with respect to the hair dye compositions containing a direct dye, excellent effects on the emulsion stability and the hair dyeing ability were observed by adding both of the component (B) and the component (D).

Moreover, in Comparative Example 3-3 and Comparative Example 3-4, a cationic water-soluble polymer having ionicity which was similar to that of the component (D) was added instead of the component (D). However, the emulsion stability and the hair dyeing ability did not improve.

Although the kind of the direct dye was changed in Example 6-2, the effects on the emulsion stability and the hair dyeing ability were maintained.

The hair dye composition of the invention can be used as a hair dye for dyeing human hair, beard, mustache, whiskers, eyebrows or body hair such as hair on the leg. Moreover, the hair dye composition may also be used for dyeing body hair of an animal such as a pet. The hair dye composition can also be used for a cosmetic method for dyeing body hair of a human or an animal at a beauty salon, a barbershop or the like.

What is claimed is:

1. A hair dye composition which is an emulsion composition containing an oily component, characterized by containing the following components (A-1) and (B):
an oxidation dye (A-1); and
a surfactant having a sterol skeleton (B).

2. The hair dye composition according to claim 1, characterized by further containing a lanolin derivative surfactant (C).

3. A hair dye composition which is an emulsion composition containing an oily component, characterized by containing the following components (A-2), (B) and (D):
a direct dye (A-2);
a surfactant having a sterol skeleton (B); and
a cationic surfactant (D).

4. The hair dye composition according to claim 1, characterized in that the surfactant having the sterol skeleton (B) is polyoxyethylene phytosterol.

5. The hair dye composition according to claim 2, characterized in that the surfactant having the sterol skeleton (B) is polyoxyethylene phytosterol.

6. The hair dye composition according to claim 3, characterized in that the surfactant having the sterol skeleton (B) is polyoxyethylene phytosterol.
